(19) United States
(12) Patent Application Publication
(10) Pub. No.: US 2026/0140037 A1
(43) Pub. Date: May 21, 2026

(54) TEST SYSTEM AND METHOD FOR DETERMINING FLUID MIGRATION FRONT BASED ON PULSE PRESSURE-ENHANCED DISTRIBUTED STRAIN

(71) Applicant: DALIAN UNIVERSITY OF TECHNOLOGY, Dalian (CN)

(72) Inventors: Lanlan JIANG, Dalian (CN); Yongchen SONG, Dalian (CN); Jindi LIU, Dalian (CN); Jintao XU, Dalian (CN); Mingjun YANG, Dalian (CN); Yi ZHANG, Dalian (CN); Lunxiang ZHANG, Dalian (CN)

(21) Appl. No.: 19/389,923

(22) Filed: Nov. 14, 2025

(30) Foreign Application Priority Data
Nov. 17, 2024 (CN) 202411638394.3

(51) Int. Cl. G01N 15/08; G01N 33/24

(52) U.S. Cl. CPC G01N 15/0826; G01N 33/24

(57) ABSTRACT

Disclosed are a test system and method for determining a fluid migration front based on pulse pressure-enhanced distributed strain, which belong to the technical field of CO₂ geological storage and oil and gas resource development. The system includes a CO₂ displacement brine unit, an optical fiber sensor, a pulse pressure injection unit, a data acquisition and processing system, and the like. By injecting high-pressure gas into the other side of a core, a large pressure difference is instantaneously generated at a two-phase interface, resulting in significant strain changes. These strain changes are monitored by the optical fiber sensor in real time, and a position of a CO₂/brine migration front is analyzed and determined by a data collecting and processing system.

### TECHNICAL FIELD

The present disclosure relates to the field of CO₂ geological storage and oil and gas resource development, and in particular, to a method and system for determining a CO₂/brine migration front in a core displacement experiment using the pulse pressure and optical fiber sensing technology.

### BACKGROUND

With the intensifying global climate change, the increasing emission of greenhouse gases, especially carbon dioxide (CO₂), is considered one of the main causes. In response to this challenge, countries worldwide are actively seeking solutions to reduce CO₂ emissions. CO₂ geological storage technology, as an effective means to reduce emission, involves injecting CO₂ into deep underground geological formations (such as saline aquifers, depleted oil and gas fields and coal seams) for long-term storage, preventing its release into the atmosphere. This technology shows great potential in mitigating global warming. Although the CO₂ geological storage technology is theoretically feasible and has potential, many challenges remain in its practical applications, especially ensuring the safety and effectiveness of the storage process. Monitoring the migration behavior of CO₂ underground, determining whether CO₂ is safely stored, and evaluating the long-term effect of storage are key steps. Effective monitoring technologies can provide important information about CO₂ diffusion paths, storage stability, and possible leakage risks, thereby providing scientific basis and technical support for the storage process.

The optical fiber sensing technology, due to the advantages of high sensitivity, strong anti-interference capability and long-distance distributed monitoring, has been widely used in CO₂ geological storage monitoring. Commonly used optical fiber sensing technologies include fiber Bragg grating, Raman scattering and Brillouin scattering, all of which can monitor the changes in temperature, strain and pressure. However, in the CO₂ storage process, due to low injection rate and other factors, stress changes in a storage area can be subtle, and it is difficult for traditional optical fiber sensors to effectively capture these minute changes, limiting their monitoring capabilities. To solve the above problems, the present disclosure proposes a method and system based on pulse pressure for determining a CO₂/brine migration front.

### SUMMARY

To solve the above problems, the present disclosure proposes a test system and method for determining a fluid migration front based on pulse pressure-enhanced distributed strain. In the method, by injecting high-pressure gas into the other side of a core, a large pressure difference is instantaneously generated at a CO₂/brine interface, resulting in significant strain changes. This method amplifies a stress change signal during the storage process, enabling an optical fiber sensor to more accurately monitor these changes and determine a position of a CO₂/brine migration front.

A technical solution of the present disclosure is as follows: a test system for determining a fluid migration front based on pulse pressure-enhanced distributed strain, including a CO₂ displacement brine unit, a pulse pressure injection unit, a confining pressure control unit, and a distributed optical fiber strain monitoring unit.

The CO₂ displacement brine unit includes an experimental core, a core holder and a CO₂/brine injection pump. The core is mounted in a dedicated core fixture to ensure tightness and stability. CO₂ and brine are respectively injected into one side of the core by an injection system to simulate a fluid migration process under an underground storage condition. Meanwhile, the injection pressure and flow rate of the CO₂ and brine are controlled to ensure that experimental conditions are controllable.

In the CO₂ displacement brine unit, a brine tank is connected to a four-way joint sequentially through a second valve, a second injection pump and a third valve, and a first CO₂ gas cylinder is connected to the four-way joint through a first injection pump and a first pressure sensor. The four-way joint is then connected to a pore pressure injection hole of the core holder through a fourth valve and a second pressure sensor to communicate with a core.

In the pulse pressure injection unit, a second CO₂ gas cylinder is connected to the four-way joint sequentially through a third injection pump, a third pressure sensor and a fifth valve.

In the confining pressure control unit, a deionized water bottle is connected to confining pressure injection holes of the core holder sequentially through a fourth injection pump, a seventh valve and a fourth pressure sensor to communicate with a confining pressure interlayer.

The distributed optical fiber strain monitoring unit includes a distributed optical fiber, an optical fiber demodulator and a computer module. The distributed optical fiber is configured for transmitting an optical signal to a data processing module, and is spirally wound and fixed onto the core to ensure that the optical fiber is evenly distributed and firmly fixed. Meanwhile, one end of the optical fiber is connected to the optical fiber demodulator to monitor stress changes on the surface of the core during the displacement process. By receiving and converting an optical signal from an optical fiber sensor, the optical fiber demodulator converts the optical signal into an electrical signal and performs amplification, filtering, spectrum analysis and demodulation on the electrical signal to extract strain data, monitoring strain changes along the optical fiber in real time. An analog-to-digital converter converts an analog electrical signal into a digital signal and transmits the digital signal to a data processing and analyzing system. The computer module further analyzes the digital signal to obtain strain distribution along the optical fiber.

Further, the system also includes a collection unit, and the core holder passes through a sixth valve via a pipeline and is then connected to a gas-water collection tank.

Further, the computer module is electrically connected with an optical fiber demodulator, and the data demodulator is electrically connected with the distributed optical fiber The distributed optical fiber is configured for transmitting a detected optical signal to the optical fiber demodulator, and a collected signal is modulated by the optical fiber demodulator to carry information about amplitude, frequency and phase. The signal is analyzed and processed by the computer module, and excitation intensities corresponding to different points on the optical fiber are displayed on a computer in real time.

A test method for determining a fluid migration front based on pulse pressure-enhanced distributed strain, including the following steps:
S1, selecting a suitable experimental core sample, spirally winding and fixing an optical fiber onto the outer surface of a core, and mounting the core in a core holder after packaging;
S2, conducting a core displacement experiment: respectively injecting CO₂ and brine into one side of the core by an injection system, and controlling the injection pressure and flow rate of the CO₂ and brine to ensure that experimental conditions are controllable;
S3, conducting pulse injection of high-pressure gas: rapidly injecting high-pressure gas into the other side of the core, instantaneously injecting high-pressure gas using a quick response valve to generate a significant pressure difference;
S4, monitoring using optical fiber strain sensors arranged around the core, and acquiring corresponding relevant data information;
S5, enabling a signal transmitted in an optical fiber cable to enter the optical fiber demodulator for demodulation; and
S6, analyzing and processing the signal by the computer module to output a strain change, where the position where the strain peak suddenly changes is the CO₂/brine migration front.

Further, the method includes the following steps:
S1, core preparation stage: performing deionized water saturation treatment on the core to ensure that no air exists in the core; arranging the distributed optical fiber on the surface of the core along a fluid migration path to ensure that the distributed optical fiber covers the entire displacement area; and spirally and tightly winding and fixing the distributed optical fiber onto the core;
S2, confining pressure control: injecting deionized water in the deionized water bottle into the confining pressure interlayer of the core holder through the confining pressure injection holes using the fourth injection pump to generate a required confining pressure; and ensuring that the core is always subjected to a uniform and stable confining pressure throughout the displacement experiment;
S3, CO₂ displacement brine stage: injecting brine in the brine pool into the core through the pore pressure injection hole by the second injection pump to ensure that pores inside the core are completely saturated; then, injecting CO₂ in the first CO₂ gas cylinder into the core using the first injection pump, and conducting a brine displacement experiment to ensure that a pore pressure does not exceed a confining pressure during the entire displacement process; and collecting the displaced brine into a gas-water collection tank;
S4, injection of pulse pressure: with the fifth valve closed, adjusting a pulse pressure to a preset pulse pressure, which is higher than an original pore pressure but lower than the confining pressure by the third injection pump, and controlling the pulse pressure to be injected into the core holder by opening and closing the fifth valve;
S5, optical fiber strain signal acquisition stage: monitoring and recording distributed optical fiber strain data during pulse pressure injection in real time, with particular attention given to strain changes at a CO₂/brine interface; extracting high-resolution strain data by the optical fiber demodulator, and analyzing and processing the data by the computer module; and visualizing the data, and generating a strain distribution map and a strain-time changing curve to identify the strain changes caused by the pulse pressure and determine a position of a CO₂/brine migration front.

Through the above-mentioned technical solution, the present disclosure has the following beneficial effects:

In conjunction with the pulse pressure and distributed optical fiber sensing technology, the present disclosure effectively solves the problems of detecting fluid migration front in core displacement experiment where the strain changes are subtle and difficult to identity, typically caused by low injection rate. By injecting high-pressure gas into the other side of a core, a large pressure difference is instantaneously generated at a CO₂/brine interface, resulting in significant strain changes. These strain changes are monitored by the optical fiber sensor in real time, and the position of the CO₂/brine migration front is analyzed and determined by the data collecting and processing system. The technology significantly improves the accuracy and sensitivity of monitoring the CO₂/brine migration front, and can provide high-resolution strain data in real time, ensuring the accuracy and reliability of experimental data, and providing powerful technical support for the research on CO₂ geological storage. The method has the advantages of high monitoring precision, rapid response, wide applicability and the like, and it can dynamically track the migration of CO₂ during the underground storage process, ensuring safety and effectiveness of the storage. Meanwhile, the present disclosure can also be applied to the monitoring of other geological fluid migration processes, such as groundwater resource management, and oil and gas field development.

### BRIEF DESCRIPTION OF THE DRAWINGS

The accompanying drawings described here are for explanatory purposes only and are not intended to limit the scope of the present disclosure in any way. In addition, the shapes and proportional dimensions of various components in the figures are merely illustrative and are intended to facilitate understanding of the present disclosure, rather than specifically limiting the shapes and proportional dimensions of the various components of the present disclosure. Under the teaching of the present disclosure, those skilled in the art can choose various possible shapes and proportional dimensions to implement the present disclosure according to specific conditions.

FIG. 1 is a schematic diagram of a test system for determining a fluid migration front based on pulse pressure-enhanced distributed strain.

FIG. 2 is a top view of a core holder.

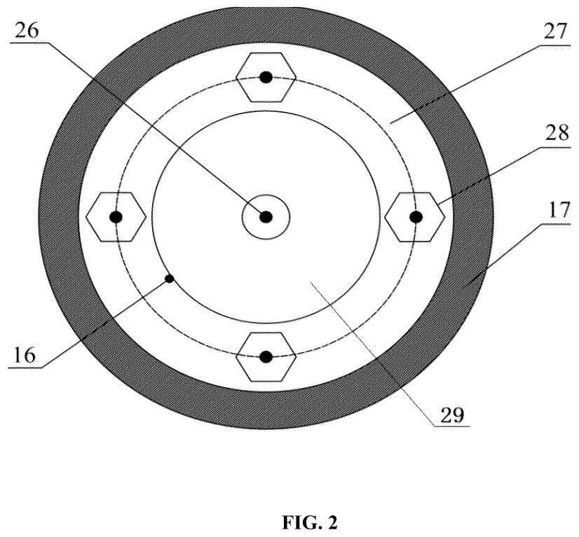

In the figures: 1. First injection pump; 2. First CO₂ gas cylinder; 3. First valve; 4. First pressure sensor; 5. Brine tank; 6. Second valve; 7. Third valve; 8. Four-way joint; 9. Fourth valve; 10. Second injection pump; 11. Fifth valve; 12. Second CO₂ gas cylinder; 13. Third pressure sensor; 14. Third injection pump; 15. Second pressure sensor; 16. Distributed optical fiber; 17. Core holder; 18. Gas-water collection tank; 19. Sixth valve; 20. Fourth pressure sensor; 21. Seventh valve; 22. Fourth injection pump; 23. Deionized water bottle; 24. Optical fiber demodulator; 25. Computer module; 26. Pore pressure injection hole; 27. Confining pressure interlayer; 28. Confining pressure injection hole; 29. Core.

### DETAILED DESCRIPTION OF THE EMBODIMENTS

In order to make those skilled in the art better understand the solutions of the present disclosure, the technical solutions in the embodiments of the present disclosure will be clearly and completely described below with reference to the accompanying drawings in the embodiments of the present disclosure.

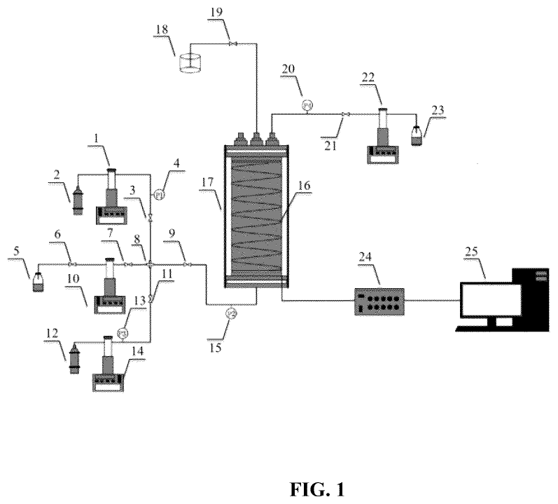

FIG. 1 shows a test system for determining a fluid migration front based on pulse pressure-enhanced distributed strain, including a CO₂ displacement brine unit, a confining pressure control unit, a pulse pressure injection unit, and a distributed optical fiber strain monitoring unit. The distributed optical fiber strain monitoring unit includes a signal transmission module, an optical fiber demodulator and a computer module.

The CO₂ displacement brine unit includes a first injection pump 1, a CO₂ gas cylinder 2, a first valve 3, a first pressure sensor 4, a brine tank 5, a second valve 6, a third valve 7, a four-way joint 8, a second injection pump 10 and a core holder 17. The first injection pump 1 is configured to inject CO₂ gas, the second injection pump is configured to inject brine, and both are connected to a pore pressure injection hole of the core holder. The pulse pressure injection unit includes a fifth valve 11, a second CO₂ gas cylinder 12, a third pressure sensor 13 and a third injection pump 14. A preset high pulse pressure is achieved through the third injection pump 14. In order to minimize the influence on the displacement experiment, CO₂ is also used as a pulse pressure medium. The confining pressure control unit includes a fourth pressure sensor 20, a seventh valve 21, a fourth injection pump 22 and a deionized water bottle 23. The distributed optical fiber strain monitoring unit includes a distributed optical fiber 16, an optical fiber demodulator 24 and a computer module 25. The distributed optical fiber 16 is configured for transmitting a detected optical signal to the optical fiber demodulator. A collected signal is modulated by the optical fiber demodulator 24 to carry key information such as amplitude, frequency and phase. The signal is analyzed and processed by the computer module 25, and excitation intensities corresponding to different points on the optical fiber are displayed on a computer in real time. The computer module 25 is connected to the optical fiber demodulator 24, and the data demodulator is connected to the distributed optical fiber 16.

In the core holder 17, the outer side of a core 29 is provided with a confining pressure interlayer 27, a pore pressure injection hole 26 is formed in the core 29, and confining pressure injection holes 28 are formed in the confining pressure interlayer 27; and the distributed optical fiber 16 is wound around the outer side of the core 29.

In actual operation, brine is first pumped from the brine pool 5 by the second injection pump 10, and then injected into the core 29 of the core holder 17 through the second valve 6, so that pores inside the core 29 are fully saturated. During the injection process, a pressure change is monitored by the first pressure sensor 4, to brine is observed flowing out from the other side to ensure that the core 29 is completely filled with brine.

Then, CO₂ is extracted from the CO₂ gas cylinder using the first injection pump 1, and injected into the core 29 for brine displacement experiment. The first valve 7 and the third valve 9 are opened to allow CO₂ to smoothly enter the core holder 17. In order to control a pressure during the injection process, the injection rate is monitored and adjusted as needed by a second pressure sensor 15 to ensure that a pore pressure does not exceed a confining pressure.

In order to enhance a strain signal during the CO₂ displacement brine process, a high pulse pressure is pre-stored by the third injection pump 14. During this process, the fifth valve 11 is kept closed, the pulse pressure is adjusted to a preset pulse pressure, which is higher than the pore pressure but lower than the confining pressure by the third injection pump 14, and the injection of the pulse pressure is controlled by rapidly opening and closing the fifth valve 11.

Throughout the whole process mentioned above, stress distribution is monitored in real time by the distributed optical fiber, with particular attention given to the strain data of the optical fiber during pulse pressure injection and observation on a strain change at a CO₂/brine interface. High-resolution strain data is extracted by the optical fiber demodulator 24 and is analyzed and visualized by the computer module 25, then a strain distribution map and a strain-time changing curve are generated.

When working with the above-mentioned technical solution, the method includes the following steps:
(1) Core preparation stage: performing deionized water saturation treatment on the core 29 to ensure that there is no air or other impurities inside the core 29; arranging the distributed optical fiber 16 on the surface of the core along a fluid migration path to ensure that the optical fiber covers the entire displacement area; spirally and tightly winding and fixing the distributed optical fiber 16 onto the core 29 to maximum strain change detection; and checking an optical fiber connection to ensure the stability and reliability of signal transmission.
(2) Confining pressure control experiment: injecting deionized water into the confining pressure interlayer 27 of the core holder 17 using the fourth injection pump 22 to generate a required confining pressure. This step ensures that the core 29 is always subjected to a uniform and stable confining pressure throughout the displacement experiment. By uniformly applying an external pressure, the confining pressure can reproduce a three-dimensional stress state of the underground rock stratum, preventing core fracturing or deformation and ensuring the accuracy and repeatability of measurement data.
(3) CO₂ displacement brine stage: injecting brine by the first injection pump 1 to ensure that pores inside the core 29 are completely saturated; then, injecting CO₂ using the second injection pump 10 to perform brine displacement experiment so as to ensure that a pore pressure does not exceed a confining pressure in the entire displacement process; and collecting the displaced brine into a gas-water collection tank 18.
(4) Injecting a pulse pressure into the system in order to enhance a strain signal generated in the CO₂ displacement brine process, ensuring that with the valve 11 closed, the pulse pressure is adjusted to a preset pulse pressure, which is higher than the original pore pressure but lower than the confining pressure by the third injection pump 14, and the injection of pulse pressure is controlled by opening and closing the valve 11.
(5) Optical fiber strain signal acquisition stage: monitoring and recording distributed optical fiber strain data during pulse pressure injection in real time, with particular attention given to strain changes at a CO₂/brine interface; extracting high-resolution strain data by the optical fiber demodulator, and analyzing and processing the data by the computer module; and visualizing the data, and generating a strain distribution map and a strain-time changing curve to identify the strain changes caused by pulse pressure and determine a position of a CO₂/brine migration front.

What is claimed is:

1. A test method for determining a fluid migration front based on pulse pressure-enhanced distributed strain, wherein a test system adopted in the test method comprises a CO₂ displacement brine unit, a pulse pressure injection unit, a confining pressure control unit, and a distributed optical fiber strain monitoring unit;
in the CO₂ displacement brine unit, a brine tank is connected to a four-way joint sequentially through a second valve, a second injection pump and a third valve, a first CO₂ gas cylinder is connected to the four-way joint through a first injection pump and a first pressure sensor, and the four-way joint is then connected to a pore pressure injection hole of a core holder through a fourth valve and a second pressure sensor to communicate with a core;
in the pulse pressure injection unit, a second CO₂ gas cylinder is connected to the four-way joint sequentially through a third injection pump, a third pressure sensor and a fifth valve;
in the confining pressure control unit, a deionized water bottle is connected to confining pressure injection holes of the core holder sequentially through a fourth injection pump, a seventh valve and a fourth pressure sensor to communicate with a confining pressure interlayer; and
the distributed optical fiber strain monitoring unit comprises a distributed optical fiber, an optical fiber demodulator and a computer module, the distributed optical fiber being spirally wound and fixed onto the core;
the test method comprises the following steps:
S1, core preparation stage;
S2, confining pressure control;
S3, CO₂ displacement brine stage: injecting brine in the brine tank into the core through the pore pressure injection hole by the second injection pump to ensure that pores inside the core are completely saturated; then, injecting CO₂ in the first CO₂ gas cylinder into the core using the first injection pump to perform a brine displacement experiment so as to ensure that a pore pressure does not exceed the confining pressure during the entire displacement process; and collecting the displaced brine into a gas-water collection tank;
S4, injection of pulse pressure: with the fifth valve closed, adjusting a pulse pressure to a preset pulse pressure higher than the pore pressure but lower than the confining pressure by the third injection pump, and controlling the pulse pressure to be injected into the core of the core holder by opening and closing the fifth valve;
S5, optical fiber strain signal acquisition stage: monitoring and recording distributed optical fiber strain data during pulse pressure injection in real time, with particular attention given to strain changes at a CO₂/brine interface; extracting high-resolution strain data by the optical fiber demodulator, and analyzing and processing the data by the computer module; and visualizing the data, and generating a strain distribution map and a strain-time changing curve to identify the strain changes caused by the pulse pressure and determine a position of a CO₂/brine migration front.

2. The test method according to claim 1, wherein the system further comprises a collection unit, and the core holder is connected to the gas-water collection tank via a pipeline through a sixth valve.

3. The test method according to claim 1, wherein the computer module is electrically connected with the optical fiber demodulator, and the optical fiber demodulator is electrically connected with the distributed optical fiber; and the distributed optical fiber is configured for transmitting a detected optical signal to the optical fiber demodulator; and
a collected signal is modulated by the optical fiber demodulator to carry information about amplitude, frequency and phase, which is analyzed and processed by the computer module, and excitation intensities corresponding to different points on the optical fiber are displayed on a computer in real time.

4. The test method according to claim 1, wherein in the step S1, the core preparation stage specifically comprises: performing deionized water saturation treatment on the core to ensure that no air exists in the core; arranging the distributed optical fiber on the surface of the core along a fluid migration path to ensure that the distributed optical fiber covers the entire displacement area; and spirally and tightly winding and fixing the distributed optical fiber onto the core.

5. The test method according to claim 1, wherein in the step S2, the confining pressure control comprises: injecting deionized water in the deionized water bottle into the confining pressure interlayer through the confining pressure injection holes using the fourth injection pump to generate a required confining pressure; and ensuring that the core is always subjected to a uniform and stable confining pressure throughout the displacement experiment.
